(19) United States
(12) Patent Application Publication
(10) Pub. No.: US 2004/0063629 A1
(43) Pub. Date: Apr. 1, 2004

(54) Treatment of damaged connective tissue

(76) Inventors: Simon Troy Humphrys, Verdun (AU); Robyn Woodward, Gawler (AU)

Correspondence Address: LARSON & TAYLOR, PLC, 1199 NORTH FAIRFAX STREET, SUITE 900, ALEXANDRIA, VA, 22314, US

(21) Appl. No.: 10/275,175

(22) PCT Filed: May 3, 2001
(86) PCT No.: PCT/AU01/00503

(30) Foreign Application Priority Data
May 3, 2000 (AU) PQ 7290

(51) Int. Cl. A61K 38/30

(52) U.S. Cl. 514/12

(57) ABSTRACT

This invention relates to a method for treating a tendon or ligament disorder, comprising the step of administration of a pharmaceutical or veterinary composition of IGF to the affected area of a subject in need of such treatment. The tendon or ligament disorder may be the result of a traumatic, exercise-related or overuse injury, or may be the result of, or associated with, a pathological condition. In preferred embodiments the traumatic or exercise-related injury is a ruptured tendon or ligament; a severed tendon or ligament; a tendon or ligament avulsion; a tendon or ligament sprain; limping or lameness; or the pathological condition is a disease, such as an inherited disease, an endocrinological or metabolic condition, an infectious disease, or a disease attributed to inflammation of the tendon, ligament or surrounding tissue. The invention further relates to compositions for the treatment of these conditions.

This invention relates to the treatment of damaged connective tissue, and in particular to the treatment of a tendon or ligament disorder. The invention further relates to compositions for use in the method of the invention.

### BACKGROUND OF THE INVENTION

All references, including any patents or patent applications, cited in this specification are hereby incorporated by reference. No admission is made that any reference constitutes prior art. The discussion of the references states what their authors assert, and the applicants reserve the right to challenge the accuracy and pertinency of the cited documents. It will be clearly understood that, although a number of prior art publications are referred to herein, this reference does not constitute an admission that any of these documents forms part of the common general knowledge in the art, in Australia or in any other country.

Damage to connective tissue is a relatively frequent and often incapacitating condition, which particularly arises from trauma and exercise related injuries to the tendons and ligaments at the extremities of the body, such as the hands and feet (Angermann and Lohmann, 1993; Maffulli et al., 1999). Damage to tendon and ligamentous tissue is extremely common, and often occurs as a result of sporting injuries sustained in sports such as football, basketball, netball and skiing. Damage to tendon and ligamentous tissue can also result from pathological conditions including inflammation, such as tendinitis. For the purposes of this specification, damage to tendons and ligaments arising from trauma, from exercise-related injuries or from pathological conditions is referred to collectively as “tendon or ligament disorders”.

The functional and structural properties of tendons and ligaments are very similar. More specifically, both tendons and ligaments are composed of dense, fibrious connective tissue made up of primarily of spindle-shaped cells called fibroblasts and of collagenous fibres (principally type I and III collagen).

Tendon and ligament disorders are slow to heal, because of the lack of vascularisation of connective tissues and the difficulty associated with reducing limb movement during healing. Damage to the tendon and ligament tissue further results in the disruption of the equilibrium between synthesis and degradation of tissue matrix, thus inhibiting the healing of the tissue. When left untreated, continued use of the damaged tendon or ligament can lead to the excessive degradation of structurally important proteoglycans and collagens by proteases, which can ultimately lead to total loss of mobility.

Injuries to connective tissue through overuse and excessive exercise are a major burden on the racehorse industry. A study of 1,087 Thoroughbred racehorses in training at 105 stables indicated an incidence of tendinitis during one racing season of 7%, with a further 6% incurring recurrent tendinitis (Rooney and Genovese, 1981). Further evidence obtained from epidemiological studies suggested that approximately 10% of all Thoroughbred racehorses in training will suffer exercise-related tendinitis (Jeffcott et al., 1982; Rossdale et al., 1985), which may require unproductive resting periods of up to 9 months during which the horse cannot race (McCullagh et al., 1979; Selway S J, 1982).

Current treatments of tendon or ligament disorders have focussed on promoting extrinsic repair, i.e. repair stimulated from outside the tissue, by supporting the injured tissue with surgical implants (Karns et al., 1994), and/or by treatment with non-steroidal anti-inflammatory drugs (Almekinders et al., 1998). Further treatments promoting extrinsic repair applied to animals include surgically minimising the tension placed on the injured tissue (desmotomy), and enhancing local inflammatory responses (Becker et al., 1998; Asheim et al., 1967; Asheim et al 1964). All of these methods of treatment have shown limited success with tendon and ligament disorders.

More recently, promoting intrinsic repair, i.e. repair from within the tissue structure, has been proposed as a form of therapy which may ultimately improve the quality and rate of injury repair. The process of intrinsic repair involves stimulating the fibroblast-like cells which reside between collagen fibrils in tendon and ligament tissue to migrate and/or proliferate into the injured site, resulting in the production and regeneration of new collagen matrix (Manske et al., 1984). Over the reparative period, cellular remodelling gradually replaces this new matrix with more mature collagen matrix. This gradual remodelling confers the mature connective tissue characteristics of greater strength and elasticity on the repaired tissue, and is characterised by improved collagen fibril alignment, parallel to the long axis of the tissue, which can be detected by ultrasound imaging (Manske et al., 1984; Mass et al., 1989; Mass et al., 1990).

Growth factors are the physiological regulators of intrinsic connective tissue repair, and in particular the insulin-like growth factors (IGF-I and IGF-II) can stimulate cell migration, cell proliferation, and synthesis of proteoglycan and type I collagen in avian and rabbit tendon cells and tendon explants in vitro (Banes et al., 1995; Abrahamsonn et al., 1996; 1997). This suggests that IGF may be involved in tendon repair in vitro. For the purpose of this specification, IGF-I, IGF-II and analogues of these growth factors are considered to be equivalent, and are referred to collectively as “IGF” or “IGFs”.

Murphy and Nixon (1997) showed that whole tendon explants taken from horses can be grown in vitro, and that the continual presence of IGF-I significantly enhanced tendon cell proliferation and type I collagen synthesis. However, these studies fail to provide any direction as to how these results may be obtained in vivo, and importantly, give no guidance as to what frequency of administration of IGF may be required in order to obtain effective repair.

In vivo studies of treatments to stimulate intrinsic repair of tendon rupture have so far been inconclusive. Conflicting results have been obtained in respect of the use of sodium hyaluronate (Gaughan et al., 1991; Foland et al., 1992). In addition, Dahlgren et al., (1999) have studied the effect of IGF-I, and failed to show positive effects in 5 out of 7 reported indices of wound repair. Thus, while it can be argued that the experimental results obtained by Dahlgren et al. showed certain improvements as a result of the added IGF, other tests showed no differences between the treated and control limbs. The experimental evidence reported in the article is contradictory at best, and thus the paper by Dahlgren et al does not clearly teach or suggest that any form of IGF treatment in any pattern of dosage and frequency would be expected to result in promoting the healing of a tendon or ligament disorder. Instead, the article represents a teaching that using a very specific IGF dosage regime, and under certain specific conditions, some parameters improved and others remained the same. Dahlgren fails to teach or suggest that the specific, infrequent administration protocol of the present invention would be successful.

Importantly, Dahlgren utilises frequent administration of the IGF-I (10 injections over 20 days), which is consistent with other studies showing that free IGF-I is rapidly complexed with at least six specific IGF binding proteins which alter its biological activity (Bassett et al., 1990), or which remove it from sites of administration (Robertson et al., 1999). Therefore, it appears that IGF bioactivity is short-lived.

In agreement with such findings, the prior art teaches towards a need for IGFs to be maintained consistently at an elevated level for their activities to be exhibited. This is especially evident in experiments in vitro, which demonstrate that IGFs achieve their maximum capacity to stimulate growth within 30 minutes of administration, whereas all responses are lost within 30 minutes of IGF removal (Ballard et al., 1981). Moreover, the extensive prior art relating to IGF effects in experimental animals teaches that the most effective method of IGF administration is through continuous administration via an osmotic pump (Lemmey et al., 1991; Tomas et al., 1996). Continuous administration of IGFs is also taught through applications in humans, where either multiple daily injections or continuous infusion are used wherever practical (Guler et al., 1989; Vlachopapadopoulou et al., 1995; Lai et al., 1997). In no case does the prior art teach that one or two injections of IGFs can produce a significant therapeutic effect. Thus there is no a priori basis for assuming that infrequent administration of IGF would be effective for treating a tendon or ligament disorder.

In addition, it has been shown that connective tissue is particularly subject to the impairment of healing due to local inflammatory processes as a result of physical trauma associated with intralesional injections (U.S. Pat. No. 5,618,516 issued Apr. 8, 1997). Thus there is still a need in the art for a method of treatment of a tendon or ligament disorder by infrequent administration of IGF. This is desirable from both the therapeutic and commercial points of view.

We have now surprisingly found an effective method of treating damaged connective tissue using infrequent administration of IGF. The delivery of IGFs using this method has not been reported previously, particularly in the treatment of tendon or ligament disorders. The particular dosage regime of the present invention (e.g. twice weekly administration or less) means that it will be possible to administer IGF on a out-patient basis, saving patients the time and expense of yet another hospitalisation while improving patient quality of life.

### SUMMARY OF THE INVENTION

In a first aspect, the invention provides a method of treatment of a tendon or ligament disorder, comprising the step of administration of an effective amount of IGF to the area of a tendon or ligament of a subject in need of such treatment.

The tendon or ligament disorder may be the result of a traumatic, exercise-related or overuse injury, or may be the result of, or associated with, a pathological condition.

In one preferred embodiment the tendon or ligament disorder is a ruptured tendon or ligament. Such a rupture may be caused by an overuse or exercise- or sports-related injury. Partial ruptures are also within the scope of invention.

In a second preferred embodiment the tendon or ligament disorder is a severed tendon or ligament. For example, the severed tendon or ligament may be a result of physical injuries sustained particularly to the extremities of the body, such as the hands and feet. Tendon or ligament severing caused by laceration, crushing or division of the tissue is specifically contemplated.

In a third preferred embodiment the tendon or ligament disorder is a tendon or ligament avulsion. For example, the tendon or ligament avulsion may be associated with a sporting or other physical injury resulting in a tendon detaching from the bone or muscle or a ligament detaching from bone or cartilage. Tendon and ligament avulsions caused by pathological conditions are also contemplated.

In a fourth preferred embodiment the tendon or ligament disorder is a tendon or ligament sprain. Such a sprain may be associated with a sporting or overuse injury, causing the tendon or ligament fibres to stretch and leading to the disruption of the tendon or ligament fibre bundles.

In a fifth preferred embodiment the tendon or ligament disorder is limping or lameness resulting in an alteration in the normal walking pattern. Preferably the treatment accelerates the time to functional use of the tendon or ligament.

In a sixth preferred embodiment the tendon or ligament disorder is a disease. Such a disease may be an inherited disease such as ochronosis, an endocrinological or metabolic condition such as diabetes mellitus, an infectious disease such as an bacterial infection, or a disease attributed to inflammation of the tendon, ligament or surrounding tissue, such as tendinitis, rheumatoid arthritis, rheumatoid tendinitis, peritendinitis or tenosynovitis.

Preferably the IGF is administered up to twice weekly; more preferably the IGF is administered less frequently than twice weekly. For example, the IGF may be administered in a single administration, or alternatively may be administered on two occasions 7 or more days apart. It will be appreciated that the term “administration” encompasses a situation in which part of the dose is given at separate sites within the areas to be treated, ie. in a divided dose.

It will be appreciated that in the method of the invention, the mammal to be treated may be a human, or may be a domestic, companion or zoo animal. For example the mammal may be a cat, dog, horse, camel or human. More preferably the mammal is a human or a horse.

In a second aspect, the invention provides a composition for treating a tendon or ligament disorder, comprising a therapeutically or veterinarily effective amount of IGF together with a pharmaceutically acceptable diluent or carrier.

In a third aspect, the invention provides a list for treatment of a tendon or ligament disorder, comprising a composition according to the invention and instructions for use of the composition in the method of the invention.

In all aspects of the invention the IGF may be IGF-I, IGF-II, or a mixture of both IGF-I and IGF-II. The IGF may be any IGF of any species. Preferably the IGF is the IGF homologue specific to each species. The IGF may be isolated from a naturally-occurring source, or it may be chemically synthesised or produced by recombinant DNA technology; preferably the IGF is recombinant. Preferably the IGF used in this invention is human or horse IGF, more preferably recombinantly-produced human or horse IGF.

It is to be clearly understood that the present invention extends to biologically active fragments or functional analogues of human IGF, i.e. analogues or derivatives of human IGF in which the wild-type IGF sequence includes additions, deletions or substitutions by another amino acid or an amino acid analogue, provided that the biological activity of the IGF is retained. IGF analogues suitable for use in the invention include those described in U.S. patents U.S. Pat. No. 5,077,276, U.S. Pat. No. 5,164,370, U.S. Pat. No. 5,470,828, and U.S. Pat. No. 5,330,971, and in International Patent Application No. PCT/AU99/00292, all assigned to GroPep Limited.

The terms “fragment”, “analogue”, and “derivative” of IGF mean a molecule which retains essentially the same biological function or activity as IGF. Thus an analogue includes a proprotein which can be activated by cleavage of the proprotein portion to produce an active mature polypeptide.

The term “treatment” as used herein is intended to include either therapeutic treatment of an existing tendon or ligament disorder, or preventive or prophylactic procedures performed before the occurrence of the disorder. Thus the patient to be treated may already have the tendon or ligament disorder, or may be at risk of having the tendon or ligament disorder. The term “treatment” also includes:

1) the regeneration of new tendon or ligament tissue, which adds to the existing tendon or ligament tissue at the onset of treatment, and which may serve to replace tendon or ligament lost prior to the onset of treatment;

2) the preservation of existing tendon or ligament tissue, which encompasses tendon or ligament tissue existing at the onset of treatment and any newly formed tendon or ligament tissue following onset of treatment; and/or

3) the acceleration of the time to functional use of a tendon or ligament.

In accordance with this invention, the IGF is administered in therapeutically effective amounts. The term “therapeutically effective amount” as used herein means that amount necessary at least partly to attain the desired effect, i.e. regeneration and/or preservation of a tendon or ligament. Such amounts will depend on the particular injury being treated, the severity of the injury, and the characteristics of the individual subject, including age, physical condition, size, weight and other concurrent treatment, and will be at the discretion of the attending physician or veterinarian. These factors are well known to those of ordinary skill in the art, and can be addressed with no more than routine experimentation. It is generally preferred that a minimum effective dose be determined according to sound medical or veterinary judgement. It will be understood by those of ordinary skill in the art that a higher dose may be administered for medical, psychological or other reasons.

Preferably the IGF is administered by localised administration. Such administration may be achieved directly at the site, for example by an intralesional injection to the damaged tendon or ligament, by topical administration of the IGF to the exposed tendon or ligament at the time of surgery, or with a delivery system. Alternatively, other modes of administration, such as systemic injections, may be used, provided that they increase the amount of IGF at the tendon or ligament tissue to attain the desired affect.

Methods and pharmaceutical carriers for the preparation of pharmaceutical compositions, including compositions for intralesional or topical administration, are well known in the art, as set out in textbooks such as Remington's Pharmaceutical Sciences, 18^{th} Edition, Mack Publishing Company, Easton, Pa. USA.

Suitable pharmaceutically acceptable carriers and/or diluents include conventional solvents, saline solutions, dispersion media, fillers, aqueous solutions, antibacterial and antifungal agents and absorption-promoting agents. Except insofar as any conventional medium or agent is incompatible with the active ingredient, its use in the pharmaceutical compositions of the present invention is contemplated. Supplementary active ingredients which have the ability to promote wound healing or to inhibit inflammation may also be incorporated into the compositions. For example, the pharmaceutical composition may additionally include one or more other cytokines, including but not limited to insulin, epidermal growth factor, fibroblast growth factor, betacellulin, transforming growth factor-α or transforming growth factor-β.

The intralesional and/or localised administrations contemplated by the present invention include administration of any formulations suitable for systemic injection of IGF, such as aqueous isotonic solutions, suspensions, gels, and polymers impregnated with IGF, or for topical administration of IGF, such as aqueous creams, ointments, gels, lotions, sprays, microspheres, liposomes, wound dressings, and synthetic polymer dressings or sutures impregnated with IGF, and the like. Preferably for topical administration the IGF is formulated in a fibrin gel.

Preferred aspects of the treatment of a tendon or ligament disorder with IGF according to the invention include the following:

(a) The IGF concentration in a solution or gel may range from 0.1 mg/ml-100 mg/ml, and is preferably 2.5-10 mg/ml.

(b) The amount of solution or gel applied is usually about 0.1 ml per discrete core lesion within the tendon or ligament. However, it will be understood by those of ordinary skill in the art that more diffuse injuries may require additional applications throughout the affected area.

(c) The recommended application frequency is twice weekly or less frequently than twice weekly. For example the IGF may be administered in a single administration, or alternatively may be administered on two occasions 7 or more days apart.

(d) Symptomatic subjects are identified after a careful clinical examination of the symptoms of a tendon or ligament disorder. This clinical examination suitably includes testing for swelling, inflammation, pain, discomfort, immobility and/or joint stiffness. Further examination would also include careful assessment of the traumatic or exercise-related injury or pathological condition underlying the disorder.

For the purposes of this specification it will be clearly understood that the word “comprising” means “including but not limited to”, and that the word “comprises” has a corresponding meaning.

### DETAILED DESCRIPTION OF THE INVENTION

The invention will now be described in detail by way of reference only to the following non-limiting examples.

### EXAMPLE 1

### IGF Formulations

Suitable formulations for use in intralesional or localised applications of IGF in accordance with this invention, and methods of preparation thereof, include the following, in which “IGF” may be IGF-I, IGF-II or an analogue thereof:

Isotonic Solution

Phosphate (10 mM) buffered saline (150 mM) stabilized with aspartate (10 mM)

IGF quantity specified (qs), dissolved in 100 mM acetic acid and diluted in phosphate buffered saline stabilized with aspartate, and stored at 4° C. until required.

Fibrin Gel

IGF qs

Thrombin solution: 0.002 g thrombin added to 1 ml medium.

Fibrinogen solution: 0.04-0.20 g added to 10 mls of 0.9% (w/v) sodium chloride which has been sterilised by membrane filtration (0.22 μm membrane) and kept warm at 37° C. Kept at 37° C. until required.

IGF qs is added to the fibrinogen solution prior to gel formation. To make gel, add thrombin to fibrinogen at a ratio of 1:10.

Collagen Gel

IGF qs

Type I/type III collagen (1%)

IGF qs is added to the collagen solution prior to gel formation.

Methylcellulose Gel

IGF qs

Methylcellulose (2%)

IGF qs is added to the methylcellulose solution prior to gel formation.

### EXAMPLE 2

### Intralesional Injection of IGF-I Promotes Intrinsic Repair of Acute Collagenase-Induced Tendinitis in Horses

Studies on the pathogenesis of equine tendinitis following collagenase injury concluded that this particular model can justifiably be used as a model of traumatic and overuse injuries (Williams et al., 1984)

12 healthy Standardbred horses with collagenase-induced tendinitis in their nearside forelimbs were used in this study. Tendinitis was induced in the superficial digital flexor tendon (SDFT), and the contralateral undamaged tendon was used as a control, with the undamaged deep digital flexor tendon (DDFT) as an additional control.

Treatments involved administration by intralesional injection of either active drug (250 μg of IGF-I formulated in 100 μl of phosphate-buffered saline) to six (6) horses or placebo (100 μl of vehicle) to the other six (6) horses on two occasions seven days apart, and monitoring the progress of tissue repair over the ensuing 8 weeks by ultrasound image analysis of samples of “core lesions”. The study investigators were blinded to the treatments.

The digitised images were used to analyse the echoic properties of the “core lesion” using a mean grey scale. This analysis correlates the proportion of reflected soundwaves (the whiteness of ultrasound images) to the proportion of connective tissue present within the “core lesion” during the healing process. Therefore mean grey scale change over time can be used as a measure of wound healing by expressing the mean cross-sectional soundwave echo of “core lesions” as degrees of whiteness. For example, the whiter the mean cross-sectional area, the more connective tissue is present, and the greater is the progression toward repaired tendon. Table 1 shows the mean gradients and standard error of the mean (SEM) gradients for the two treatment groups, and the statistical significance (p) of IGF-I treatment, as determined from repair rates generated by image analysis of core lesions. The means were calculated from individual values of the mean grey scale of the treated core lesion within the superficial digital flexor tendon (SDFT), expressed as a percentage of the undamaged deep digital flexor tendon (DDFT), normalised to the contralateral undamaged tendon.

Linear regression curves generated from the mean grey scale data analysis illustrated in Table 1 demonstrate significantly steeper gradients for horses treated with IGF-I compared to control horses.

TABLE 1
Effect of IGF-I on healing response

| | | Mean gradient | |
|---|---|---|---|
| | Group | (healing response) | SEM |
| 1. | Vehicle treated | 25.7 | 5.02 |
| 2. | IGF-I treated | 44.5 | 3.75 |

t value = 3.00
p value = 0.0134
n = 12

The proportion of type I and type III collagen in injured tendon was also determined. Tissue from core lesions was excised 15 weeks after tendinitis was induced, dried, frozen and ground to a fine powder under liquid nitrogen in a SPEX machine. Ground tissue was dissolved in 10× volume (v/w) 0.5M acetic acid containing Complete™ protease inhibitors (Roche) and digested with pepsin (10:1 tissue:pepsin w/w) overnight at 4° C. with constant mixing. Solutions were clarified by centrifugation (45 minutes at 30,000 g) and the supernatant transferred to new vials. Ice-cold 4M NaCl was added to the supernatant to a final concentration of 2M NaCl, mixed by inversion and incubated on a rotary mixer for 30 minutes at 4° C. Collagenous proteins were pelleted by centrifugation (45 minutes at 30,000 g) and the supernatant poured off. The collagenous proteins were redissolved in 10 ml of 0.5M acetic acid and dialysed overnight against 0.5M acetic acid at 4° C. with stirring. Collagen types were resolved using a C18 column, 300 Å pore size (250×4.6 mm) and elution times of type I and type III standards were recorded as references.

The analysis of this data was complicated by the standards resolving into multiple peaks, with some overlap between type I and type III peak elution times. To confirm HPLC peaks containing type I and type III collagen, peaks were collected and characterised using SDS-PAGE (6% Tris-Glycine). From this analysis the area under respective type III and type I collagen peaks was integrated, and proportions of each collagen type calculated for each tendon. The proportion of type I collagen was expressed as a percentage of the total type I and type III collagen.

The proportion of type I collagen was significantly increased compared to control horses, as shown in Table 2, which compares the percentage of type I collagen in the two treatment groups. Table 2 shows the mean percentage of type I collagen and standard error of the mean (SEM) percentages for type I collagen from the two treatment groups, and the statistical significance (p) of IGF-I treatment.

TABLE 2
Effect of IGF-I on type I collagen content of tendon

| | | Mean type I | |
|---|---|---|---|
| | Group | collagen percentage | SEM |
| 1. | Vehicle-treated | 51.2 | 4.16 |
| 2. | IGF-I treated | 69.0 | 4.2 |

t value = 3.28
p value = 0.0083
n = 12

The means were calculated from individual values for each horse, as determined by HPLC analysis of collagen in core lesion samples. The percentages were determined by calculating the area under the curves of HPLC chromatographic traces for the core lesion sample from each horse's tendon, compared to type I and type III collagen references. An increase in the proportion of type I collagen compared with type III collagen is indicative of more functionally mature tendon.

These two significant results indicate that IGF-I treated equine tendinitis heals more rapidly than untreated tendinitis, as shown by the steeper gradients of linear regression for mean grey scale analysis, and more closely resemble mature uninjured tendon in their proportion of type I collagen, which also suggests higher quality repair of tendon tissue treated with IGF-I.

### EXAMPLE 3

### Intralesional Injection of IGF-I Promotes Intrinsic Repair of Acute Collagenase-Induced Tendinitis in Horses; Comparisons between Doses and Frequency of IGF-I Administration

24 healthy horses with collagenase-induced tendinitis in their nearside forelimbs were used in this study. Tendinitis was induced in the same way as described in Example 2.

Treatments involved administration by intralesional injection of either

(1) 250 μg of IGF-I formulated in 100 μl of phosphate-buffered saline on each of two occasions seven days apart,

(2) 500 μg of IGF-I formulated in 100 μl of phosphate-buffered saline on each of two occasions seven days apart,

(3) a single injection of 500 μg of IGF-I formulated in 100 μl of phosphate-buffered saline followed by 100 μl of vehicle seven days later, or

(4) 100 μl of vehicle alone on each of two occasions seven days apart.

There were six (6) horses in each group.

The ultrasound procedure was carried out as described in Example 2, and the mean grey scale of the treated core lesion within the superficial digital flexor tendon expressed as a percentage of the undamaged deep digital flexor tendon determined at 2, 4, 6 and 8 weeks following creation of the lesion. These values are shown in Table 3 as means ±SEM for the six horses in each group at each time period.

TABLE 3
Effect of IGF-I on healing response

| | Mean ± at: | | | |
|---|---|---|---|---|
| Group | 2 weeks | 4 weeks | 6 weeks | 8 weeks |
| 1. (IGF-I, 2 × 250 μg) | 73.1 ± 1.8 | 81.4 ± 2.8 | 80.6 ± 3.4 | 88.1 ± 1.4 |
| 2. (IGF-I, 2 × 500 μg) | 67.6 ± 2.5 | 81.7 ± 1.5 | 80.8 ± 2.7 | 86.2 ± 1.7 |
| 3. (IGF-I, 1 × 500 μg) | 69.2 ± 2.2 | 80.8 ± 3.9 | 77.2 ± 2.4 | 85.1 ± 1.8 |
| 4. Vehicle Treated | 68.1 ± 2.9 | 72.9 ± 1.2 | 72.7 ± 2.8 | 73.8 ± 4.7 |

Regression curves generated from the mean grey scale data analysis in Table 3 demonstrate steeper gradients for horses treated with any of the three IGF-I protocols than for the vehicle group. No differences were evident between the three IGF-I protocols indicating that a dose twice that used in Example 2 (group 2, Example 3) gave no additional benefit. Importantly, a single injection of IGF-I was as effective as when two treatments of IGF-I were administered 7 days apart.

The percentage of collagen as type 1 collagen in the lesion area was measured as described in Example 2, except that biopsies were obtained 12 weeks after tendinitis was induced. Table 4 demonstrates that each of the IGF-I treated horse groups had a higher percentage of type 1 collagen than observed in the vehicle-treated group.

TABLE 4
Effect of IGF-I on type 1 collagen content of tendon

| | Mean type 1 collagen | |
|---|---|---|
| Group | percentage | SEM |
| 1. (IGF-I, 2 × 250 μg) | 58.9 | 1.8 |
| 2. (IGF-I, 2 × 500 μg) | 57.3 | 2.0 |
| 3. (IGF-I, 1 × 500 μg) | 60.4 | 3.2 |
| 4. Vehicle Treated | 51.7 | 1.4 |

As with the healing response data in Table 3, no significant differences were found between the three IGF-I groups.

Throughout the treatment and post-treatment phases the lameness of each horse was graded, using an external examination based on a clinically-accepted scale of 0 to 5, ranging from grade 0 (completely sound) to grade 5 (non-weight bearing lameness). Two independent blinded examiners evaluated horses being trotted and walked on hard level ground, and graded the horse's lameness based on this scale.

TABLE 5
Lameness regression slopes in the four horse groups

| Group | Lameness Slope | SEM |
|---|---|---|
| 1. (IGF-I, 2 × 250 μg) | −0.45 | 0.04 |
| 2. (IGF-I, 2 × 500 μg) | −0.42 | 0.07 |
| 3. (IGF-I, 1 × 500 μg) | −0.41 | 0.05 |
| 4. Vehicle Treated | −0.26 | 0.07 |

The study demonstrates higher negative slopes for the three groups of IGF-I-treated horses than for the vehicle group, indicating a more rapid return to normality in the three IGF-I-treated groups. As with the healing response and the percentage of type 1 collagen in the lesion area, there were no differences between the three IGF-I-treated groups.

### EXAMPLE 4

### Methodologies for Characterisation of IGF-I Treatment for Severed Tendons

The person skilled in the art will readily be able to investigate the use of the invention to promote healing of a severed tendon or ligament, for example using the severed tendon model in chickens.

Suitable chickens for this model include the species Gallus domestious. Preferably, the birds are anaesthetized with an intramuscular injection, and a foot is washed and then swabbed with a 10% povidone-iodine solution. Using aseptic techniques, an incision 1-1.5 cm long is made through the plantar surface of the third digit, starting halfway along the first pad distal to the toe webbing, and the subcutaneous fat is cut using iridectomy scissors until the tendon sheath is visible. Synovial fluid will escape when the sheath is cut to expose the long digital flexor tendon (LDFT) as it emerges between the branches of the intermediate flexor tendon (IFT). To maintain a high moisture content, the surgical field can be irrigated with sterile physiological (0.9%) saline. The LDF tendon is raised, and a suture is passed transversely, right to left, through the tendon close to the bifurcation of the IFT; Maxon-CV®, 6-0, polyglyconate, monofilament suture is suitable. A modified Kessler stitch is used to appose the two ends of the transected tendon.

The IGF composition is applied between the two tendon surfaces. Preferably the IGF is formulated in a fibrin gel. The suture is then tied with even tension, taking care not displace the IGF composition. The tendon sheath is closed with an interrupted stitch, followed by closure of the skin with a 4-0 silk suture. The toe is wrapped with adhesive bandage, and to prevent rubbing the bandage is placed on the front of the foot and leg up to the knee joint. The chicken's foot can then be placed in a fibreglass cast specifically designed to maintain the toes straight and bent in flexion approximately 25° at the metatarsal/phalangeal joint. Cotton wool is used to pack the toes lightly within the cast, which is then tapped to the leg. Finally, intramuscular injections of buprenorphine hydrochloride (0.03 mg/kg) and amoxicillin/clavulanic acid (0.2 ml/kg) are administered, and the birds are allowed to recover from the anaesthetic.

At a suitable time following the treatment, preferably five weeks after treatment, the chickens are anaesthetised and then euthanased by the administration of 120 mg/kg of sodium pentobarbitone. Various methods are known in the art for assessment and characterisation of the effectiveness of healing of a severed tendon. For example, histology and tensiometry may be used. For histological assessment, preferably the flexor tendon is exposed and freed from the sheath and any excessive adhesive tissue. A piece of tendon approximately 3 centimetres long, evenly spaced each side of the transection, is held flat using biopsy pads inside a histology cassette, then placed into buffered formalin for 48 hours. The tendon is then transferred to 70% alcohol prior to processing and sectioning. For the assessment of cell density, cellular alignment, neutrophil number and fibroblast alignment, 5 μm sections are stained with haematoxylin/eosin. Adjacent 5 μm sections are stained with Masson's trichrome stain, which demonstrates the supporting tissue elements, principally collagen. Preferably these qualitative measurements are performed independently and in blinded fashion. For the tensiometry assessment, the left chicken leg is removed at the knee joint on a saline-soaked swab and frozen immediately at −20° C. until all samples for the group have been collected. Legs are thawed at 4° C. overnight and kept on ice prior to tensiometry. The flexor tendon is removed, measured and the breaking strain tested, for example using a 250N load cell on a Mecmesin tensiometer.

On the basis of the results shown in example 2 and 3, the inventors expect that the invention used in this particular model would accelerate the healing of the severed tendon injury and accelerate time to functional use of the tendon as measured by tensiometry, cell density, cellular alignment, neutrophil number, fibroblast alignment and/or lameness scoring.

### EXAMPLE 5

### Methodologies used to Study the Effectiveness of IGF-I Treatments for Ligament Injuries in Dogs

The invention may be used to treat a tendon or ligament avulsion. The person skilled in the art will readily be able to investigate the claimed invention to treat a tendon or ligament avulsion.

For example, the ligament avulsion model in dogs may be used for the characterisation of the claimed invention for treating ligamental avulsions. The ligament which stabilises the lateral surface of a dog's toe commonly tears away from the bone at one of its sites of attachment during exercise. This veterinary condition in dogs is commonly referred to as “sprung toe”. Current practice involves a wide variety of treatments, including limb immobilisation and surgical re-attachment with sutures. In cases undergoing surgical repair accelerated ligament attachment to the bone is preferred, resulting in a higher quality of repair and leading to a greater chance of functional recovery.

Suitable dogs for this model include the racing greyhound, in which ligament avulsion is common due to excessive exercise. Preferably, an IGF-I formulation is applied to the site of surgical re-attachment prior to suturing the ligament end into place.

For example, the dogs are anaesthetised and the injured foot prepared for surgery. Contained in an aseptic field, the injured ligament is exposed through an incision in the lateral side of the toe. Once exposed, the damaged end of the ligament is excised and removed. The site of original attachment is examined and surgically prepared, followed by the direct application of an appropriate IGF-I formulation. Preferably the IGF is formulated in a fibrin gel. The surgically prepared ligament end is approximated to its previously prepared original site of attachment and the ligament is initially held in place by a figure of eight suture pattern. On completion of the growth factor treatment and surgical repair, the incision site is sutured and closed, and the dog's foot splinted and bandaged.

Preferably, following a period of recuperation of up to 3 months, the extent of healing and the relative effectiveness of IGF-I at promoting ligament reattachment is characterised. Various methods are known in the art to assess and characterise the effectiveness of healing of a ligament avulsion, including physical examination, histology, tensiometry and walking pattern assessment. For example, examination by palpation may be used.

On the basis of the results shown in example 2 and 3, the inventors expect that the invention used in this particular model would accelerate the healing of the ligamental avulsion injury and accelerate time to functional use of the ligament as measured by palpation, histology, tensiometry and/or lameness scoring.

It will be apparent to the person skilled in the art that while the invention has been described in some detail for the purposes of clarity and understanding, various modifications and alterations to the embodiments and methods described herein may be made without departing from the scope of the inventive concept disclosed in this specification.

References cited herein are listed on the following pages, and are incorporated herein by this reference.

REFERENCES

Abrahamsson S O, Lohmander S. Differential effects of insulin-like growth factor-I on matrix and DNA synthesis in various regions and types of rabbit tendons. J Orthop Res 1996 May;14(3):370-6.

Abrahamsson S O. Similar effects of recombinant human insulin-like growth factor-I and II on cellular activities in flexor tendons of young rabbits: experimental studies in vitro. J Orthop Res 1997 March; 15(2):256-62.

Almerkinders L C. Tendinitis and other chronic tendinopathies. J Am Acad Orthop Surg 1998;6:157-64.

Angermann, P. and Lohmann, M. Injuries to the hand and wrist. A study of 50 272 injuries. J Hand Surgery [Br] 1993 October; 18(5): 642-4

Asheim A, Knudsen O. Percutaneous tendon splitting in horses. Proceedings Am Assoc Equine Pract 1967;255-262.

Asheim A. Surgical treatment of tendon injuries in the horse. J Am Vet Med Assoc 1964;145:446-451.

Ballard F J, Nield M K, Francis G L, Knowles S E. Regulation of intracellular protein degradation by insulin and growth factors. Acta Biol Med Germ 1981;40:1293-1300.

Banes A J, Tsuzaki M, Hu P, Brigman B, Brown T, Almekinders L, Lawrence W T, Fischer T. PDGF-BB, IGF-I and mechanical load stimulate DNA synthesis in avian tendon fibroblasts in vitro. J Biomech 1995 December;28(12):1505-13

Bassett N S, Breier B H, Hodgkinson S C, Davis S R, Henderson H V, Gluckman P D. Plasma clearance of radiolabelled IGF-1 in the late gestation ovine fetus. J Dev Physiol 1990 August; 14(2):73-9

Becker C K, Savelberg H H, Buchner H H, Barneveld A. Long-term consequences of experimental desmotomy of the accessory ligament of the deep digital flexor tendon in adult horses. Am J Vet Res 1998;59:347-51.

Dahlgren L. A., Nixon, A. J. Bertram, J. E. A., van de Meulen, M. C. H., Starrak, G. S. Insulin-like growth factor I improves cellular and molecular aspects of healing in a collagenase-induced model of flexor tendinitis. 45^{th} Annual Meeting, Orthopaedic Research Society, February 1-4, Anaheim, Calif.

Foland J W, Trotter G W, Powers B E, Wrigley R H, Smith F W. Effect of sodium hyaluronate in collagenase-induced superficial digital flexor tendinitis in horses. Am J Vet Res 1992; 53: 2371-2376.

Gaughan E M, Nixon A J, Krook L P, Yeager A E, Mann K A, Mohammed H, Bartel D L. Effects of sodium hyaluronate on tendon healing and adhesion formation in horses. Am J Vet Res 1991; 52:764-773.

Guler H P, Schmid, Zapf J, Froesch E R. Effects of recombinant insulin-like growth factor I on insulin secretion and renal function in normal human subjects. Proc Natl Acad Sci USA 1989;86:2868-2872.

Jeffcott L B, Rossdale P D, Freestone J. An assessment of wastage in Thoroughbred racing from conception to four years of age. Equine Vet J 1982; 14:185-198

Karns D J, Heidt R S Jr, Holladay B R, Colosimo A J. Case Report: Revision anterior cruciate ligament reconstruction. Arthroscopy 1994;10:148-51.

Lia E C, Felice K J, Festoff B W, Gawel M J, Gelinas D F, Kratz R, Murphy M F, Natter H M, Noris F H, Rudnicki S A, and the North Amercia ALS/IGF-I Study Group. Effect of recombinant human insulin-like growth factor-I on progression of ALS. Neurology 1997;49:1621-1630.

Lemmey A B, Martin A A, Read L C, Tomas F M, Owens P C, Ballard F J. IGF-I and the truncated analogue des(1-3)IGF-I enhance growth in rats after gut resection. Am J Physiol 1991;260:E213-E219.

Maffulli N, Waterston S W, Squair J, Reaper J and Douglas A S. Changing incidence of Achillies tendon rupture in Scotland: a 15 year study. Clin J Sport Med 1999 July; 9(3): 157-160

Manske P R, Gelberman R H, Vande Berg J S, et al. Intrinsic flexor tendon repair. A morphological study in vitro. J Bone Joint Surg 1984;66:385-396.

Mass D P, Tuel R J. Human flexor tendon participation in the in vitro repair process.

J Hand Surg (Am) 1989;14;64-71.

Mass D P, Tuel R J. Participation of human superficialis flexor tendon segments in repair in vitro. J Orthop Res 1990;8:21-34.

McCullagh K G, Goodship A E, Silver I A. Tendon injuries and their treatment in the horse. Vet Rec 1979; 105:54-57.

Murphy D J, Nixon A J. Biochemical and site specific effects of insulin-like growth factor I on intrinsic tenocyte activity in equine flexor tendons. Am J Vet Res 1997; 58:103-109.

Robertson J G, Belford D A, Ballard F J. Clearance of IGFs and insulin from wounds: effect of IGF-binding protein interactions. Am J Physiol 1999 April;276(4 Pt 1):E663-71.

Rooney J R, Genovese R L. A survey and analysis of bowed tendon in Thoroughbred racehorses. J Equine Vet Sci 1981; 1:49-53.

Rossdale P D, Hopes R, Wingfield-Digby N J. Epidemiological study of wastage among racehorses in 1982 and 1983. Vet Rec 1985; 116:66-69.

Soslowsky L J, Carpenter J E, DeBano C M, Banerji I, Moalli M R. Development and use of an animal model for investigations on rotator-cuff disease. J Shoulder Elbow Surg 1996 September-October; 5(5):383-92.

Tomas F M, Lemmey A B, Read L C, and Ballard F J. Superior potency of infused insulin-like growth factor-I analogs which bind poorly to IGF-binding proteins is maintained when administered by injection. J Endocrinol 1996; 150: 77-84.

Vlachopapadopoulou E, Zachwieja J J, Gertner J M, Manzione D, Beir D M, Mattews D E, Slonim A E. Metabolic and clinical response to recombinant human insulin-like growth factor-I in myotonic dystrophy—a clinical research centre study. J Clin Endo Metab 1995;80:3715-3723.

Williams I F, McCullagh K G, Goodship A E and Silver I A. Studies on the pathogenesis of equine tendinits following collagenase injury. Res Vet Sci 1984 May;36(3):326-38.

1. A method of treating a tendon or ligament disorder, comprising the step of administration of a pharmaceutical or veterinary composition of IGF to the affected area of a subject in need of such treatment.

2. A method according to claim 1, in which the tendon or ligament disorder results from a traumatic, exercise-related or overuse injury.

3. A method according to claim 2, in which the traumatic or exercise-related injury is a ruptured tendon or ligament.

4. A method according to claim 2, in which the traumatic or exercise-related injury is a severed tendon or ligament.

5. A method according to claim 2, in which the traumatic or exercise-related injury is a tendon or ligament avulsion.

6. A method according to claim 2, in which the traumatic or exercise-related injury is a tendon or ligament sprain.

7. A method according to claim 2, in which the traumatic or exercise-related injury is lameness or limping.

8. A method according to claim 1, in which the tendon or ligament disorder is, or results from, a pathological condition.

9. A method according to claim 8, in which the pathological condition is an inherited disease, an endocrinological or metabolic condition, an infectious disease, or a disease attributed to inflammation of the tendon, ligament or surrounding tissue.

10. A method according to claim 9, in which the inherited disease is ochronosis.

11. A method according to claim 9, in which the endocrinological or metabolic condition is diabetes mellitus.

12. A method according to claim 9, in which the infectious disease is a bacterial infection.

13. A method according to claim 8, in which the disease attributed to inflammation of the tendon, ligament or surrounding tissue is tendinitis, rheumatoid arthritis, rheumatoid tendinitis, peritendinitis or tenosynovitis.

14. A method according to claim 9 or claim 13, in which the pathological condition is tendinitis.

15. A method according to any one of claims 1 to 14, in which the subject is a mammal.

16. A method according to claim 15, in which the mammal is a cat, a dog, a horse or a human.

17. A method according to claim 16, in which the subject is a horse.

18. A method according to claim 16, in which the subject is a human.

19. A method according to any one of claims 1 to 18, in which the IGF is
(a) IGF-I, IGF-II, or an IGF analogue, or
(b) a mixture of two or more of the IGFs set out in (a).

20. A method according to any one of claims 1 to 19, in which the IGF is a human IGF.

21. A method according to any one of claims 1 to 20, in which the IGF is a recombinant IGF.

22. A method according to any one of claims 1 to 21, in which the composition is administered by intralesional injection.

23. A method according to any one of claims 1 to 21, in which the composition is administered topically to an exposed tendon or ligament.

24. A method according to any one of claims 1 to 23, wherein the administration frequency is twice weekly or less.

25. A method according to claim 24, in which the IGF is administered in a single administration.

26. A method according to claim 24, in which the IGF is administered on two occasions 7 or more days apart.

27. A method according to any one of claims 24 to 26, in which the administration is given as a divided dose.

28. A method according to any one of claims 1 to 27, in which the treatment alleviates or prevents the symptoms of a tendon or ligament disorder.

29. A method according to any one of claims 1 to 27, in which the treatment delays the onset of, inhibits the progression of, or halts altogether, the onset or progression of a tendon or ligament disorder.

30. A composition for treating a tendon or ligament disorder, comprising a therapeutically effective amount of IGF together with a pharmaceutically or veterinarily acceptable diluent or carrier, in which the treatment comprises the step of administration of a pharmaceutical or veterinary composition of IGF to the affected area of a subject in need of such treatment, and in which the composition is adapted to intralesional or topical administration.

31. A composition according to claim 30, in which the IGF is IGF-I, IGF-II, or an IGF analogue, or a mixture thereof.

32. A composition according to any one of claims 1 to 22, in which the IGF is a human IGF.

33. A composition according to any one of claims 30 to 32 in which the IGF is a recombinant IGF.

34. A composition according to any one of claims 30 to 33, comprising an amount of IGF effective to alleviate or prevent the symptoms of a tendon or ligament disorder.

35. A composition according to any one of claims 30 to 34, comprising an amount of IGF effective to delay the onset of, inhibit the progression of, or halt altogether, the onset or progression of a tendon or ligament disorder.

36. A composition according to any one of claims 30 to 35, additionally comprising one or more other cytokines or other agents.

37. A composition according to claim 36, in which the other cytokine is insulin, epidermal growth factor, fibroblast growth factor, betacellulin, transforming growth factor-α or transforming growth factor-β.

38. A composition according to any one of claims 30 to 37, which is a formulation selected from the group consisting of aqueous creams, ointments, gels, lotions, sprays, microspheres, liposomes, wound dressings, and synthetic polymer dressings or sutures impregnated with IGF.

39. A composition according to any one of claims 30 to 38, in which the IGF is formulated in collagen or fibrin gel.

40. Use of IGF for the manufacture of a medicament for treating a tendon or ligament disorder, in which the treatment comprises the administration of a pharmaceutical or veterinary composition of IGF to the affected area of a subject in need of such treatment

41 Use according to claim 40, wherein the administration frequency is twice weekly or less.

42. Use according to claim 40 or claim 41, in which the IGF is IGF-I, IGF-II, or an IGF analogue, or a mixture thereof.

43. Use according to any one of claims 40 to 42, in which the IGF is a human IGF.

44. Use according to any one of claims 40 to 43, in which the IGF is a recombinant IGF.

45. Use according to any one of claims 40 to 44, in which the treatment alleviates or prevents the symptoms of a tendon or ligament disorder.

46. Use according to any one of claims 40 to 45, in which the treatment delays the onset of, inhibits the progression of, or halts altogether, the onset or progression of a tendon or ligament disorder.

47. A kit for treatment of a tendon or ligament disorder, comprising
a) a composition comprising a therapeutically effective amount of an IGF and a pharmaceutically or veterinarily acceptable carrier; and
b) instructions for use of the composition in a method of treatment according to any one of claims 1 to 29.

48. A kit according to claim 46, in which the composition is as defined in any one of claims 30 to 39.
